(19) United States
(12) Patent Application Publication
(10) Pub. No.: US 2022/0338476 A1
(43) Pub. Date: Oct. 27, 2022

(54) Non-toxic, alcohol-free disinfectant and sanitizing composition

(71) Applicants: José Santiago, Dorado, PR (US); Angel Reinaldo Santiago Figueroa, Bayamon, PR (US)

(72) Inventors: José Santiago, Dorado, PR (US); Angel Reinaldo Santiago Figueroa, Bayamon, PR (US)

(21) Appl. No.: 17/238,826

(22) Filed: Apr. 23, 2021

(51) Int. Cl. A01N 37/36; A01N 25/30; A01N 25/04; A01N 33/12; A61K 8/92; A61K 8/41; A61K 8/60; A61K 8/365; A61Q 17/00

(52) U.S. Cl. CPC A01N 37/36; A01N 25/30; A01N 25/04; A01N 33/12; A61K 8/922; A61K 8/416; A61K 8/604; A61K 8/365; A61Q 17/005; A61K 2800/5422

(57) ABSTRACT

A non-toxic, alcohol-free disinfectant and sanitizing composition comprising a non-ionic surfactant system, an alkylated vegetable oil, a sodium-containing base, benzalkonium chloride, citric acid and purified water for use on skin, hands and hard surfaces. The materials from which it is manufactured are all renewable, non-toxic, alcohol-free, nonflammable and the composition is readily biodegradable. The composition does not rely upon alcohol to produce its antimicrobial effects. The invention provides effective alternative to alcohol-based disinfectant and sanitizers and may be particularly useful in personal care and cleaning household products for schools, hospitals, restaurants, hotels and supermarkets. Furthermore, the invention also met the Environmental Protection Agency (EPA) standards and protocols for disinfectants on hard surfaces.

### STATEMENT REGARDING FEDERALLY SPONSORED RESEARCH AND DEVELOPMENT

N/A

### RELATED APPLICATIONS

N/A

### BACKGROUND OF THE DISCLOSURE

### Technical Field

The present invention relates to cleaning compositions, and more particularly, to disinfectant and sanitizer compositions for use on skin, hands and hard surfaces. The present invention comprises a non-ionic surfactant system, an alkylated vegetable oil, a sodium-containing base, benzalkonium chloride, citric acid and purified water. The present disclosure provides a non-toxic, alcohol-free, disinfectant and sanitizer composition having environmental benefits and exhibiting effective antimicrobial activity. More particularly, the invention also met the Environmental Protection Agency (EPA) standards and protocols for disinfectants on hard surfaces.

### Discussion of the Background

Alcohol-based disinfectant and sanitizers compositions (60%-70% alcohol) are commonly used to reduce or eliminate the transmission of pathogenic organisms in skin or surface. However, alcohol-based disinfectant and sanitizers, although effective in rapidly killing germs, disrupt the skin's moisture and pH balance by stripping away the natural oils, leaving the skin dry, cracked, and more susceptible to infections. Further, alcohol-based disinfectants and sanitizers compositions with 60%-70% alcohol are highly flammable and can be misused with toxic consequences for the environment and health. If cleaning properties are also desired the compositions can include surfactants (generally non-ionic surfactants) and water to dilute the germ-killing chemicals to a safe user level.

Cleaning compositions (which are different from disinfectant and sanitizing compositions) are also commercially important products used in removing dirt and grime from surfaces, especially those characterized as “hard surfaces”. Hard surfaces are those which are frequently encountered in lavatories and kitchens and include, for example, toilets, bathtubs, sinks, as well as countertops, walls, floors, amongst others.

As new viruses and pathogens continues to emerge as Covid-19, there is a continuing desire for cleaning compositions which are also disinfectants and sanitizers which contain no alcohol to avoid skin damage, flammability, and environmental harmful effects.

### SUMMARY OF THE DISCLOSURE

In accordance with the principles of the current disclosure, the present exemplary embodiment is directed toward disinfectant and sanitizer compositions, and particularly to “green”, renewable, alcohol-free and biodegradable disinfectant and sanitizer compositions and methods of making the compositions. Such liquid disinfectant and sanitizer composition comprises a non-ionic surfactant system, an alkylated vegetable oil, a sodium-containing base, benzalkonium chloride, citric acid and purified water.

The composition provides excellent antimicrobial activity (99.9% at a contact time of 10 minutes) against Escherichia coli (ATCC 8537), Salmonella enterica (ATCC 14028), Pseudomona aeruginosa (ATCC 9027), Staphylococcus aureus (ATCC 6538) and Human coronavirus (strain 229E, ATCC VR-740).

Therefore, the composition is an effective alternative to alcohol-based disinfectant and sanitizers and may be particularly useful in personal care and cleaning household products for schools, hospitals, restaurants, hotels and supermarkets.

In one embodiment, the disinfectant and sanitizer composition comprises from 0.25% to 1% of a non-ionic surfactant system; about 3% to 6% by weight of an alkylated vegetable oil; about 0.5% to 2% by weight of a sodium-containing base; about 1% to 5% by weight of benzalkonium chloride; about 1% to 5% by weight of citric acid; and the balance of the composition is purified water.

### DETAILED DESCRIPTION

The presently claimed and disclosed inventive concepts provide a liquid disinfectant and sanitizer compositions comprising a no-ionic surfactant system, an alkylated vegetable oil, a sodium-containing base, benzalkonium chloride, citric acid and purified water.

The non-ionic surfactant system comprises alkyl mono- and polyglycoside surfactant having the general formula: RO(G)ₙ wherein R is a monovalent organic radical containing from about one to about 30 carbon atoms.

In one embodiment of the present invention, the polyglycosides have both short and long alkyl chain lengths. This can be accomplished by mixing two or more nonionic surfactant products having different average alkyl chain lengths. For example, a suitable surfactant mixture can contain polyglycosides having alkyl chain length of 8 carbon atoms and a second polyglycoside having alkyl chain length of 14 carbon atoms.

In one embodiment of the present invention, the nonionic surfactant system is a mixture of polyglycosides having alkyl chain lengths of from about 8 carbon atoms up to about 14 carbon atoms as disclosed in U.S. Pat. No. 8,455,426 by Shell.

The addition of a solvent to the composition improves its efficiency. Suitable solvents having “green” properties include alkylated vegetable oils. Nonlimiting examples of suitable vegetable oils include castor oil, coconut oil, corn oil, cottonseed oil, hemp oil, mustard oil, olive oil, palm oil, peanut oil, rapeseed oil (canola), rice bran oil, safflower oil, sesame oil, soybean oil, and sunflower seed oil.

Oils and fats are often distinguished based on their melting point; oils are liquid at room temperature, and fats are solid. The term “vegetable oil,” as used herein and in the appended claims, refers to any oil or fat obtained from plants, and that is liquid at room temperature in its alkylated form.

In one embodiment, the alkylated vegetable oil is an alkyl soyate. Suitable alkyl soyate solvents include, but are not limited to, methyl soyate, ethyl soyate and propyl soyate. An alkyl soyate is an alkyl ester derived from soybean oil. Soybean oil is a mixture of long chain saturated and unsaturated acids such as palmitic, stearic, oleic, and linoleic acids. Hence, the alkyl soyate includes, for instance, the methyl or ethyl ester of R—COOH.

In one embodiment, the alkyl soyate solvent is methyl soyate. Methyl soyate is produced by reacting soybean oil and methanol. It is a commonly used biodiesel in the United States.

A sodium-containing base is used to increase the pH of the composition. Also, it serves as a binding agent that keeps together all the other elements of the composition, thus providing stability and homogeneity to the composition. Suitable examples of a sodium-base include sodium carbonate and sodium hydroxide.

Benzalkonium chloride for use in the present disclosure may be specially manufactured or may be produced from combinations of commercially available benzalkonium chloride compositions. Benzalkonium chloride has been widely used in antimicrobial and disinfectant products and remain popular because they have strong biocidal activity, they are stable over a large pH range, they have low toxicity, and they are cost effective.

Surprisingly, benzalkonium chloride provides a compatible interaction with the other elements of the composition, which enhances its antimicrobial activity.

Citric acid has been used as disinfectant in home, health care and industrial settings. However, and most important, citric acid is used in the present invention to regulate and stabilize the pH range of the composition. Thus, creating a stable composition.

Purified water as used in the present invention means water that has been purified under three consecutive processes: double pass reverse osmosis, carbon filtered and UV disinfection. Known methods of reverse osmosis, filtration and UV disinfection are applied to the water. The purified water used in the present invention is free from organic and inorganic compounds and antimicrobial activity (less than 100 ppm of contaminants). After obtaining the purified water, it has to be used to prepare the composition within 48 hours.

In one embodiment, sodium carbonate is mixed with purified water at a rate of 30 hertz for 1 hour in Tank A to form a first mixture. The density of the first mixture is about 1.2 g/L and 1.8 g/L.

In Tank B, a short-chain polyglucoside is mixed with a long-chain polyglucoside at a rate of 50 hertz for 1 hour to form a non-ionic surfactant system. The pH of the non-ionic surfactant system is about 9 and 13. Methyl soyate is then mixed with the non-ionic surfactant system at a rate of 50 hertz for 45 minutes to form a second mixture. The density of the second mixture is about 1.4 g/L and 1.8 g/L.

Then, the first mixture in Tank A is mixed with the second mixture in Tank B at a rate of 60 hertz for 1 hour to form a third mixture. The pH of the third mixture is about 10 and 12 and the density between 1.08 g/L and 1.15 g/L.

Then, 5% by weight of citric acid is added to the third mixture for 60 minutes to form a fourth mixture.

Finally, 1% by weight of benzalkonium chloride is mixed to the fourth mixture for 3 hours. The composition is allowed to rest for at least 24 hours.

The present invention shows strong antimicrobial activity and it works just as well in low temperatures up to 33° F.

The antimicrobial compositions described and claimed herein may be used for a variety of purposes such as surfaces, floors, glass and windows, hard surfaces, and others. Also, the compositions described and claimed herein may be used in hands and skin. Because the compositions are nontoxic they are particularly useful for home, schools, hospitals, restaurants, hotels, supermarkets and personal care.

In order to further illustrate the present invention, the following examples are given. However, it is to be understood that the examples are for illustrative purposes only and are not to be construed as limiting the scope of the invention.

### Example 1

In one embodiment of the present invention, the disinfectant and sanitizing composition comprises: 1% by weight of polyglycosides; 5% by weight of methyl soyate; 1% by weight of sodium carbonate; 1% by weight of benzalkonium chloride; 3% by weight of citric acid; and 89% by weight of purified water.

The composition of the present invention was tested and proved to be effective against human coronavirus and other pathogens.

Experimental Tests and Results

All bacteria from stock culture were plated onto Tryptic Soy Agar and incubated at 35° C. for 24 hours. After incubation, a bacterial suspension was prepared for the bacteria by taking one 10 μL loop of the test bacteria into 10 mL of Normal Saline 0.85% until a 108 solution of cells was created with initial concentrations as follows: E. coli with a 1.9×108 concentration, P. aeruginosa with a 3.8×108 concentration, S. enterica with a 2.6×108 concentration, and S. aureus with a 5.9×108 concentration.

Sterile glass slide carriers were inoculated with 100 μL of each of the microorganism's suspensions and incubated to dry to at 35° C. for 40 minutes. Inoculated test carriers were individually sprayed with the Example 1 of the present invention, exposed for 10 minutes, and then neutralized in 10 mL of TSB with LP 80 neutralizing broth. All tests were performed in triplicates along with three untreated controls for the bacteria tested. Serial dilutions of treated-neutralized and untreated-controls of bacterial cultures were plated using Tryptic Soy Agar and incubated 35° C. for 24 hrs. Colonies were counted following incubation and used to calculate percent reduction (efficacy) of the Example 1 against the tested bacteria.

For the Human coronavirus, strain 229 E, ATCC VR-7 40, the test virus was propagated by inoculating the virus into cell culture flasks containing the appropriate host cell line and incubating at the appropriate conditions. Once the cell culture flasks displayed approximately 75-100% cytopathic effect (as determined by microscopic evaluation), the flasks were subjected to freeze-thaw cycles to release virus from infected cells. The contents of the cell culture flasks were collected and centrifuged in order to remove the cell debris. The test virus was then aliquoted and stored at <˜70° C. On the day of testing, an aliquot of the virus stock suspension was removed from cryostorage and thawed for use in the assay. The test virus contained 2% fetal bovine serum (FBS) organic soil load. The test virus was adjusted to contain 5% FBS organic soil load by adding 0.030 ml FBS to 0.970 ml test virus.

The present invention proved to be 99.9% effective against the above-mentioned pathogens at a contact time of 10 minutes and therefore, meeting the Environmental Protection Agency (EPA) standards and protocols for disinfectants on hard surfaces.

Tables

TABLE 1
Control Results

| Type of Control | E. coli | P. aeruginosa | S. enterica | S. aureus |
|---|---|---|---|---|
| Purity Control | Pure | Pure | Pure | Pure |
| Viability Controls | 210,000 | 1,620,000 | 295,000 | 36,000 |

| | |
|---|---|
| Neutralizing Subculture | No Growth |
| Medium Sterility Control | |
| Carrier Sterility Control | No Growth |

TABLE 2
Efficacy of Example 1 against E. coli.

| | Initial | | Log | |
|---|---|---|---|---|
| Replicate | Population (B) | T = 10 mins | Reduction | % Reduction |
| 1 | 16,000 | <10 | | |
| 2 | 12,000 | <10 | | |
| 3 | 21,000 | <10 | | |
| Average | 49,000 | <10 | 3.69 | 99.98% |

TABLE 3
Efficacy of Example 1 against P. aeruginosa.

| | Initial | | Log | |
|---|---|---|---|---|
| Replicate | Population (B) | T = 10 mins | Reduction | % Reduction |
| 1 | 2,270,000 | <10 | | |
| 2 | 1,600,000 | <10 | | |
| 3 | 1,850,000 | <10 | | |
| Average | 1,906,666 | <10 | 5.28 | 99.99% |

TABLE 24
Efficacy of Example 1 against S. enterica.

| | Initial | | Log | |
|---|---|---|---|---|
| Replicate | Population (B) | T = 10 mins | Reduction | % Reduction |
| 1 | 810 | <10 | | |
| 2 | 2,500 | <10 | | |
| 3 | 32,000 | <10 | | |
| Average | 11,770 | <10 | 3.07 | 99.91% |

TABLE 5
Efficacy of Example 1 against S. aureus.

| | Initial | | Log | |
|---|---|---|---|---|
| Replicate | Population (B) | T = 10 mins | Reduction | % Reduction |
| 1 | 8,000 | <10 | | |
| 2 | 17,000 | <10 | | |
| 3 | 49,000 | <10 | | |
| Average | 24,667 | <10 | 3.39 | 99.95% |

TABLE 6
Efficacy of Example 1 against Human coronavirus.

| | | Neutralization Control |
|---|---|---|
| | | Example 1 |
| Cell Control | | OOOO |
| Dilution | 10⁻¹ | ++++ |
| | 10⁻² | ++++ |
| | 10⁻³ | ++++ |
| | 10⁻⁴ | OOOO |
| | 10⁻⁵ | OOOO |
| | 10-⁻⁶ | OOOO |

| | |
|---|---|
| TCID₅₀ per 0.1 ml | 3.50 log₁₀ |

Key: + = Virus recovered; O = Virus not recovered and/or no cytotoxicity observed; T = Cytotoxicity observed

What is claimed is:

1. A disinfectant and sanitizing composition comprising of:
a nonionic surfactant system;
an alkylated vegetable oil;
a sodium-containing base;
benzalkonium chloride;
citric acid and
purified water.

2. The composition of claim 1 wherein the non-ionic surfactant system comprises a short-chain polyglucoside mixed with a long-chain polyglucoside.

3. The composition of claim 1 wherein the alkylated vegetable oil is methyl soyate.

4. The composition of claim 1 wherein the sodium-containing base comprises sodium carbonate.

5. A disinfectant and sanitizing composition comprising of:
a mixture of a short-chain polyglucoside mixed with a long-chain polyglucoside;
methyl soyate;
sodium carbonate;
benzalkonium chloride;
citric acid and
purified water.

6. The composition of claim 5 wherein the mixture of the short-chain polyglucoside mixed with the long-chain polyglucoside is about 0.25% to 1%;
methyl soyate is about 3% to 6%;
sodium carbonate is about 0.5% to 2%;
benzalkonium chloride is about 1% to 5%;
citric acid is about 1% to 5%; and
purified water is about 81% to 94.25%.

7. The process to synthesize a disinfectant and sanitizing composition comprising the steps of:
mixing sodium carbonate with purified water to form a first mixture;
mixing a short-chain polyglucoside with a long-chain polyglucoside to form a non-ionic surfactant system;
mixing methyl soyate with the non-ionic surfactant system to form a second mixture;
mixing the first mixture with the second mixture to form a third mixture;
mixing citric acid to the third mixture to form a fourth mixture; and
mixing benzalkonium chloride with the fourth mixture to form the composition.
